(19) United States
(12) Patent Application Publication
(10) Pub. No.: US 2025/0065147 A1
(43) Pub. Date: Feb. 27, 2025

(54) SYSTEMS AND METHODS FOR PHOTODYNAMIC THERAPY

(71) Applicant: Virginia Commonwealth University, Richmond, VA (US)

(72) Inventors: Seung Duk Lee, Glen Allen, VA (US); Kush Savsani, Richmond, VA (US)

(21) Appl. No.: 18/809,461

(22) Filed: Aug. 20, 2024

Related U.S. Application Data
(60) Provisional application No. 63/659,982, filed on Jun. 14, 2024.
(60) Provisional application No. 63/534,209, filed on Aug. 23, 2023.

(51) Int. Cl. A61N 5/06; A61K 41/00; A61P 35/00

(52) U.S. Cl. CPC A61N 5/062; A61K 41/008; A61N 5/0601; A61P 35/00; A61N 2005/063; A61N 2005/0652

(57) ABSTRACT

Methods for treating liver cancer in a patient are provided. The methods include steps of administering at least one dose of a therapeutically effective amount of a composition comprising indocyanine green (ICG) and applying a PDT light source to the liver of the patient, wherein the PDT light source comprises a transarterial fiber optic coaxial catheter comprising a laser light, a sheet having an array of light-emitting diode (LED) lights, or a micro-injectable LED implant configured for delivery inside the liver, wherein the PDT light source is applied for a time period sufficient to release reactive oxygen species from the photosensitizing agent and induce apoptosis of cancer cells. A PDT system may include a composition comprising ICG and a PDT device comprising at least one light source enabled for application of light to a target tumor.

### CROSS REFERENCE TO RELATED APPLICATIONS

This application claims the benefit of U.S. Provisional Patent Applications 63/534,209 filed on Aug. 23, 2023 and 63/659,982 filed on Jun. 14, 2024, the complete contents of which are herein incorporated by reference.

### FIELD OF THE INVENTION

The invention is generally related to systems and methods for treating liver cancer. In particular, the method utilizes photodynamic therapy wherein the cancer cells are first sensitized with indocyanine green (ICG).

### BACKGROUND OF THE INVENTION

Non-resectable/non-transplantable cases of hepatocellular carcinoma (HCC) (more than 70% of HCC patients) are treated with various modalities like trans-arterial embolization, radiofrequency/microwave ablation, external beam radiation, systemic chemotherapy or combinations thereof. Given there is no specific target agent for HCC due to various genetic mutations, systemic chemotherapy with immunotherapy and a VEGF inhibitor improves median survival by only several months and is not considered curative. Regarding multiple or superficial lesions with poor liver functions or severe cirrhosis, currently there is no effective treatment. Although diagnostic imaging accuracy remains a barrier to effective detection of HCC, recent progress in using indocyanine green (ICG) as an imaging adjunct is notable. ICG has long been utilized safely in the planning of surgical resections of liver tumors using ICG 15-minute clearance retention rate. ICG through the venous system moves to the liver parenchyma and excretes to the biliary system quickly. If liver function is not good, ICG retains in the blood even after 15 min. In the case of high level of ICG-15 min, hepatectomy is considered risky due to poor liver function. Recently, ICG-specific intraoperative infrared imaging has become popular in the era of laparoscopic and robotic surgery to visualize HCC. This effect is relying on the relative lack of ICG excretion by malignant liver cells compared to the surrounding parenchyma due to destruction of the biliary system inside the tumor. Despite advances in imaging liver cancer cells, there still remains a need for effective therapies.

### SUMMARY

The present disclosure utilizes ICG to both visualize cancer cells and as a photosensitizer for photodynamic therapy (PDT). Once a tumor containing ICG is exposed to a specific wavelength of light, the released reactive oxygen species (oxygen radicals, peroxides, and superoxides) can kill these cells through apoptosis, sparing the non-malignant parenchyma. PDT stands out from conventional therapies due to its minimal invasiveness, desired convenience, flexibility, potent efficacy, and high patient compliance, which provide it with important clinical significance. Thus, ICG is a safe and appropriate target agent, particularly for patients having multiple lesions with poor liver function or severe cirrhosis.

An aspect of the disclosure provides a method for treating liver cancer in a patient in need thereof, comprising administering at least one dose of a therapeutically effective amount of a composition comprising ICG, wherein cells of the cancer that take up the ICG are sensitized to a PDT light source, and applying a PDT light source to the liver of the patient, wherein the PDT light source comprises a transarterial fiber optic coaxial catheter comprising a laser light, a sheet having an array of light-emitting diode (LED) lights, or a micro-injectable LED implant configured for delivery inside the liver, wherein the PDT light source is applied for a time period sufficient to release reactive oxygen species from the photosensitizing agent and induce apoptosis of cancer cells.

In some embodiments, the ICG is bound to an additional photosensitizing agent. In some embodiments, the pharmaceutical composition is administered intravenously or intraperitoneally. In some embodiments, the PDT device is the transarterial fiber optic coaxial catheter. In some embodiments, the laser light has a wavelength of 805-815 nm. In some embodiments, the at least one dose of ICG is at least 5 mg/kg. In some embodiments, the at least one dose of ICG is administered daily from one to five days. In some embodiments, PDT is applied to the liver at least 24 hours after administration of a first dose of ICG. In some embodiments, the liver cancer is hepatocellular carcinoma.

Another aspect of the disclosure provides a PDT system, comprising a composition comprising ICG, and a PDT device comprising at least one light source enabled for application of light to a target tumor, wherein the PDT device is a transarterial fiber optic coaxial catheter comprising a laser light, a sheet having an array of LED lights, or a micro-injectable LED implant configured for delivery inside the liver. In some embodiments, the at least one light source has a wavelength of 805-815 nm.

### BRIEF DESCRIPTION OF THE DRAWINGS

FIG. 1. Diagram of photodynamic therapy according to some embodiments of the disclosure.

FIG. 2. Graph of laser wavelength that liver-ICG can absorb with maximum intensity.

FIG. 3. A transarterial fiber optic coaxial catheter according to some embodiments of the disclosure.

FIG. 4. An LED sheet according to some embodiments of the disclosure.

FIG. 5. A micro-injectable LED implant according to some embodiments of the disclosure.

FIG. 6. Diagram of use of micro-injectable LED implant according to some embodiments of the disclosure.

FIG. 7. Diagram of photodynamic therapy according to some embodiments of the disclosure.

### DETAILED DESCRIPTION

Embodiments of the disclosure provide systems and methods for photodynamic therapy (PDT) of liver cancer in a patient in need thereof. PDT is a process whereby light of a specific wavelength or waveband is directed toward a target cell or cells that have been rendered photosensitive through the administration of a photo-reactive, photo-initiating, or photosensitizing agent. This photo-reactive agent has a characteristic light absorption waveband and may be administered to a patient via intravenous injection, oral administration, or by local delivery to the treatment site. Abnormal cells in the body may selectively absorb certain photo-reactive agents to a greater extent than normal for healthy cells. Once the abnormal cells have absorbed and/or molecularly joined with the photo-reactive agent, the abnormal cells can then be treated by exposing those cells to light of an appropriate wavelength or waveband that substantially corresponds to the absorption wavelength or waveband of the photo-reactive agent.

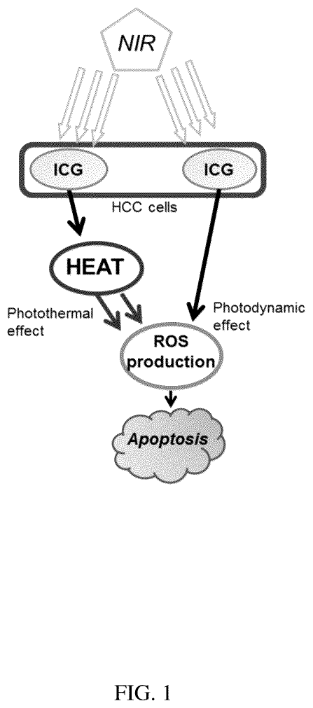

As shown in FIG. 1, the wavelength of light delivered to the targeted cells treated with the photo-reactive agent causes the agent to undergo a photochemical reaction with oxygen in the localized targeted cells, to yield free radical species (such as singlet oxygen), which cause localized cell lysis or necrosis.

Embodiments provide the use of indocyanine green (ICG) as a standalone photosensitizer which eliminates the need for additional sensitizers, streamlining the treatment process. In alternative embodiments, the ICG is bound to an additional photosensitizing agent such as porphine, chlorins, and phthalocyanine derivatives.

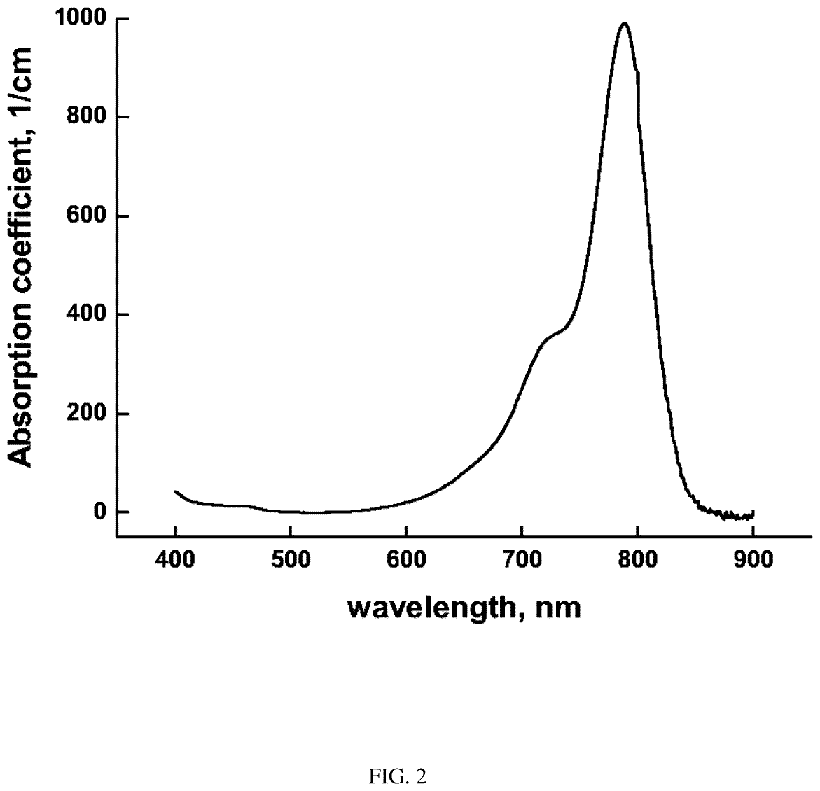

As demonstrated herein, the optimal laser wavelength that liver-ICG can absorb with maximum intensity may range from about 805-815 nm, e.g. about 809 nm (FIG. 2). Several devices which emit light at the aforementioned wavelength may be utilized. For example, the light emitter can be a light source, such as a light-emitting diode (“LED”), laser diode, organic light-emitting diode (“OLED”), or the like, or can be a terminus of a light transmission element, such as an optical fiber.

Generally, the light delivery systems can be positioned relative to a target site and then activated to deliver light to the target site. The light delivery systems can be used to treat organs (e.g. liver), vasculature, tissue (e.g., epithelial tissue, connective tissue, muscle tissue and nerve tissue), and various systems including, but not limited to, organ systems, circulatory systems, and other suitable systems in the patient. The systems and methods of the present disclosure are particularly suited for treatment of liver cancers such as hepatocellular carcinoma (HCC). In some embodiments, the HCC is an advanced HCC with poor liver function (e.g. Child-Pugh class C). Other metastatic tumors of livers may also be treated with the disclosed methods as the biliary system is destroyed around the tumor and ICG is retained inside or around the tumor.

Several parameters such as laser power (e.g. 30 J-300 J), exposure duration (e.g. 3-15 minutes), intensity (e.g. 300 W/m²), frequency, (e.g. 1-3 times per week) and ICG dose may be varied depending on the treatment required. It has been demonstrated herein that injected ICG is confined to a liver tumor after about 24 hours. Thus, in some embodiments, treatment methods include administering at least one dose of a therapeutically effective amount of a composition comprising ICG, wherein cells of the cancer that take up the ICG are sensitized to a PDT light source, and applying a PDT light source to the liver of the patient, e.g. at least about 24 hours, e.g. 24-72 hours, after administering the ICG. In some embodiments, at least one dose of ICG (e.g. about 1-4 doses) is administered daily from one to five days before PDT. The ICG dose may be administered intravenously, intraperitoneally, or orally. Compositions containing ICG are known and commercially available. Compositions as described herein may be prepared either as liquid solutions or suspensions, or as solid forms such as tablets, pills, granules, capsules, powders, ampoules, and the like. The liquid may be an aqueous liquid. Solid forms suitable for solution in, or suspension in, liquids prior to administration may also be prepared.

After ICG is administered, the PDT light source is applied for a time period sufficient to release reactive oxygen species from the photosensitizing agent and induce apoptosis of cancer cells. For example, the light source may be applied for 1-200 minutes per session. The light source may be applied over several sessions, e.g. 1-5 sessions daily, or every 2-7 days, or every 1-12 weeks.

Embodiments of the disclosure provide PDT devices useful for the methods described herein. In particular, the disclosure provides a transarterial fiber optic coaxial catheter comprising a laser light, a sheet having an array of LED lights, and a micro-injectable LED implant configured for delivery inside the liver. The particular device used may be selected based on factors such as the location and size of the tumor.

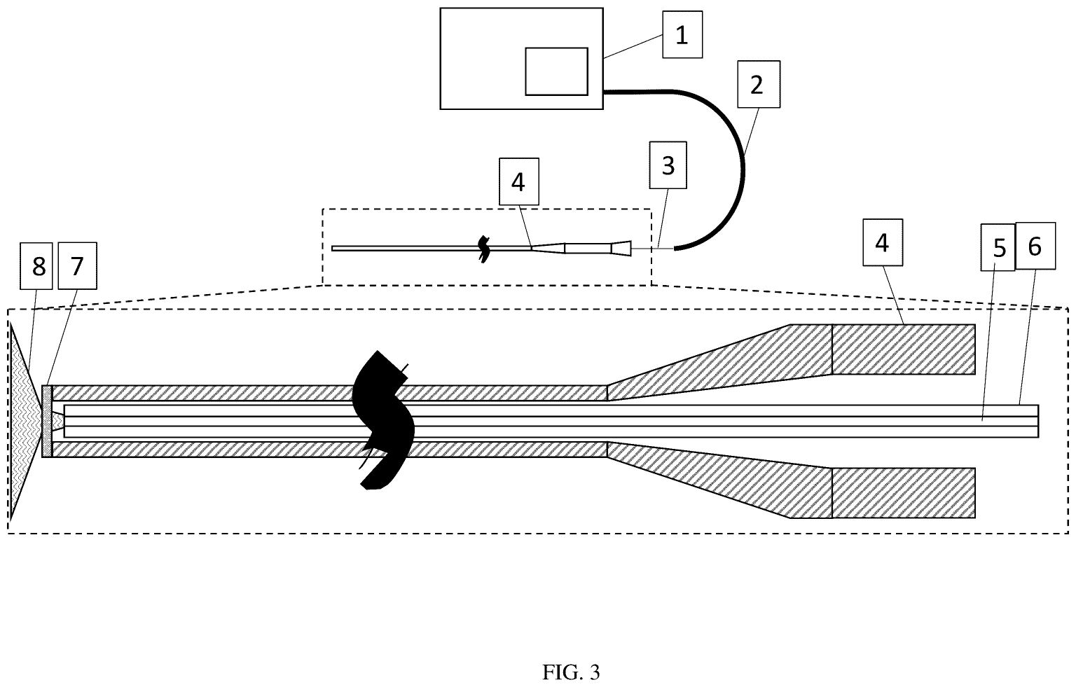

For deep seated tumors, light can be delivered via a transarterial fiber optic device which can be advanced through blood vessels to the site of the tumor. An example transarterial device is shown in FIG. 3. The device includes a laser source (1), protected fiber (2), bare fiber (3), coaxial catheter (4), bare fiber core (5), bare fiber cladding (6), diffuser (7), and laser light output (8). Transarterial fiber optic delivery is a minimally invasive technique employed to access and interrogate vasculature supplying a neoplastic mass. A slender, flexible guidewire is introduced into the arterial system, e.g. via a peripheral access point. Utilizing fluoroscopic imaging modalities, the guidewire is navigated through the arterial anatomy until it reaches the target vessel supplying the tumor. This positioning enables direct visualization and interaction with the tumor vasculature. A microcatheter is then sheathed over the guidewire to the site of the tumor and the guidewire is removed. The fiber optic is then placed through the catheter to the site of the tumor. The fiber optic component facilitates the transmission of light, allowing for application of the PDT. The catheter 4 can have a cross-sectional width that is less than about 1.25 mm, e.g. 0.5 mm to 1.25 mm.

In some embodiments, the transarterial fiber optic device can be used as an adjunct during another medical procedure, such as minimally invasive procedures, open procedures, semi-open procedures, or other surgical procedures that preferably provide access to a desired target region. Many times, the access techniques and procedures used to provide access to a target region can be performed by a surgeon and/or a robotic device, such as robotic systems used for performing minimally invasive surgeries. Optionally, the transarterial fiber optic device is used with guidewires, delivery sheaths, delivery devices (e.g., endoscopes, bronchoscopes, optical instruments, etc.), introducers, trocars, biopsy needle, or other suitable medical equipment. If the target treatment site is at a distant location in the patient, delivery devices should be used for convenient navigation through tortuous body lumens or other anatomical structures in the patient. The flexible transarterial fiber optic device can be easily positioned within the patient using, for example, steerable devices, such as endoscopes, bronchoscopes, and the like. Semi-rigid or rigid transarterial fiber optic devices can be delivered using trocars, access ports, rigid delivery sheaths using semi-open procedures, open procedures, or other delivery tools/procedures that provide a somewhat straight delivery path, for example. Advantageously, the transarterial fiber optic device can be sufficiently rigid to displace internal tissue to help facilitate light delivery to the target tissue. When inserted in the patient, the system can be easily rotated and advanced axially while maintaining its configuration.

Various materials can be used to construct the device including rubber, composite materials, thermoplastics, polymers (e.g., polyester, polyethylene terephthalate (PET), polypropylene, polyethylene naphthalate (PEN), and combinations thereof.

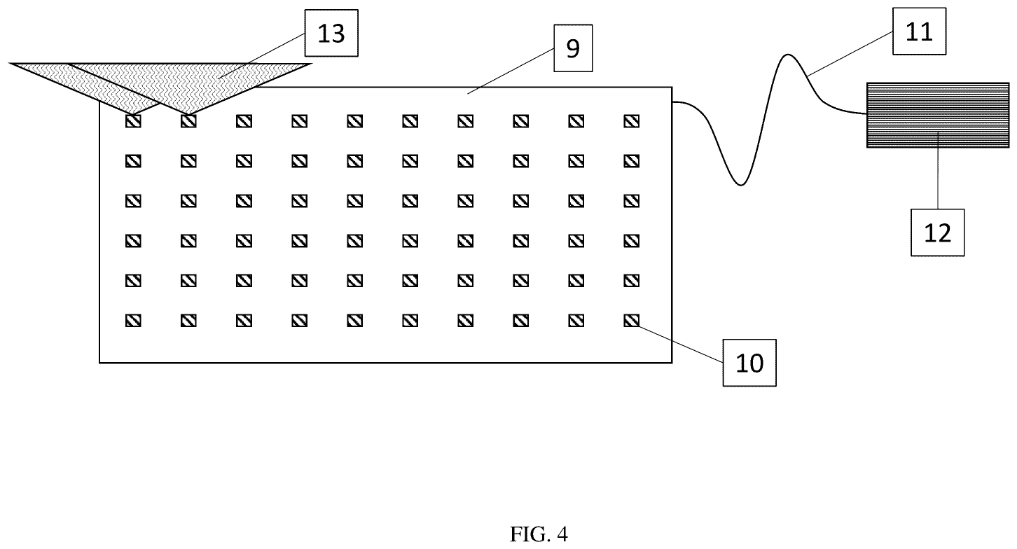

In other embodiments, the disclosure provides a sheet with multiple LEDs for PDT. As shown in FIG. 4, the sheet may comprise a flexible circuit panel (9), LEDs (10), electrical connection (11), power supply (12), and LED light output (13). The sheet may be of a size sufficient to cover and treat an entire side of the liver at one time. In alternative embodiments, the sheet is smaller thus requiring multiple positionings in order to treat the entire surface of the liver. The sheet can be delivered to the abdominal cavity with a laparoscopic procedure making small holes on the abdomen. Under endoscopic visualization, the sheet may be draped over the liver surface. Secure fixation of the LED array may be achieved utilizing standard tissue-adhesion techniques. The power source for the LEDs resides on the outside of the body and can be activated with a switch. Once the LED sheet is positioned over the liver, the light can be turned on and off with a remote controller. Once therapy is done, the LED sheet can be removed with another minimally invasive surgery.

The LEDs 10 can be arranged in parallel, series, or combinations thereof. For example, some LEDs can be arranged in series while other LEDs are arranged in parallel. As such, various circuit configurations can be used when mounting the LEDs to the sheet. Each LED can be configured be to emit the same wavelength or waveband. However, LEDs having different wavelengths or wavebands can be used to provide varying outputs. These LEDs can be activated simultaneously or at different times depending on the desired treatment. The various LEDs can also be activated and deactivated in a pulsed sequence. Alternately, the system may be programmed to selectively activate and deactivate different selected segments of LEDs. In this manner, a treatment protocol, for example causing the LEDs to be lit in a certain sequence, at a particular power level for a selected period of time, may be programmed into the control system. The sheet can have any number of LEDs, e.g. 30-100 LEDs.

Any suitable mounting means can be employed to temporarily or permanently couple the LEDs onto the sheet. For example, adhesives, bonding material, fasteners, solder, or other coupling means can securely couple the LEDs to the sheet. The flexible sheet is positioned at the treatment site and flexibly conforms to a surface within the patient's body, so that the light emitted by the plurality of light sources is directed into tissue at the treatment site. The sheet can have any shape, e.g. can be square, rectangular, or may be non-quadrilateral in shape. The sheet may be fabricated from a polymer selected for its flexible characteristics i.e., its ability to be folded over without damage and undue resistance. To protect the sheet from mechanical damage and from exposure to body fluids, the sheet may be encapsulated within a biocompatible envelope that is optically transparent.

Because of its flexible nature, the sheet can readily be inserted transcutaneously into a patient's body while rolled up, e.g., by insertion through a laparoscopic guide tube or using other conventional laparoscopic techniques, and the rolled or folded flexible sheet can then be maneuvered into position for administering light to the treatment site. Once positioned adjacent the treatment site, the rolled or folded flexible sheet is unrolled or unfolded and spread over the surface of the organ or other treatment site to which the light will be administered.

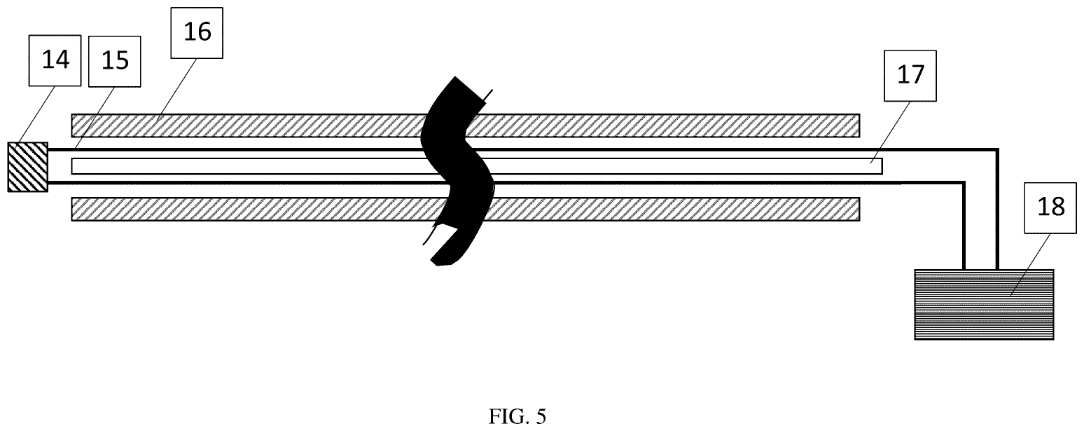

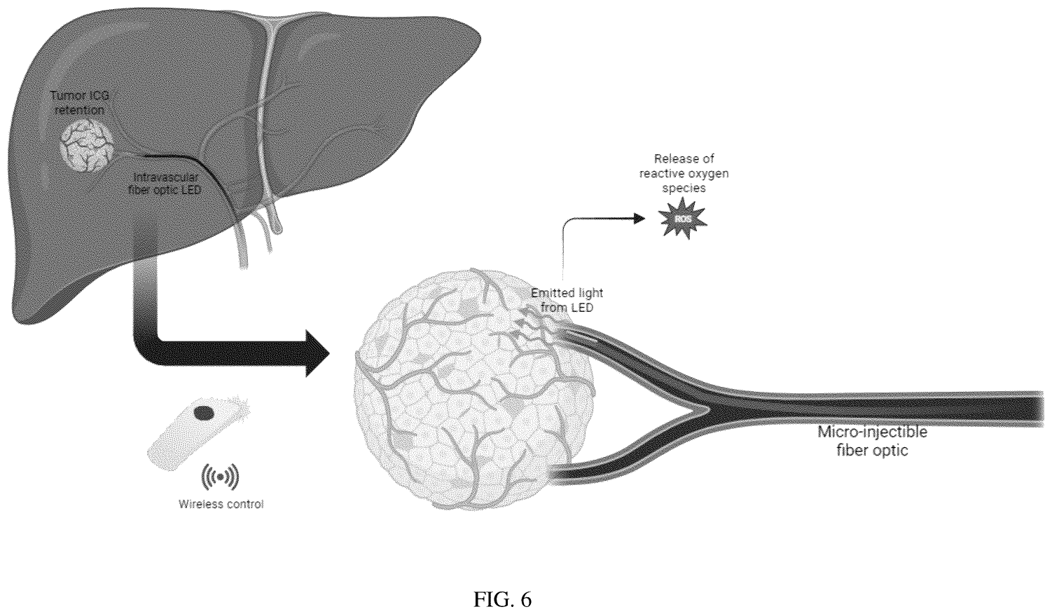

Further embodiments provide a micro-injectable LED device for administering PDT. As shown in FIG. 5, the device may comprise micro LEDs (14), electrical leads (15), needle (16), support rod (17), and power supply (18). This device is particularly suitable if the tumor is located in a deep area of liver. As shown in FIG. 6, a micro LED module can be delivered to a tumor inside the liver with a device like an injectable needle, e.g. via percutanous injection. This micro LED module can be turned on/off with a remote controller. Under CT guidance, the tumor site is located and needle access is obtained followed by LED injection. In some embodiments, the micro LED module stays inside the tumor during the therapy, and is not removable. The power for the LED is housed on the outside of the body and can be turned on and off via a switch.

For each of the devices described herein, there may be an internal or external power supply or source, such as a battery (e.g. a lithium battery). In other embodiments, the device is powered by an AC power source, such as an electrical outlet. The system can include a power cord that can be connected to the AC power source.

A patient or subject to be treated by any of the compositions or methods of the present disclosure can mean either a human or a non-human animal including, but not limited to dogs, horses, cats, rabbits, gerbils, hamsters, rodents, birds, aquatic mammals, cattle, pigs, camelids, and other zoological animals.

In some embodiments, the formulation or active agent is administered to the subject in a therapeutically effective amount. By a “therapeutically effective amount” or an “effective amount” is meant a sufficient amount to treat the disease or disorder at a reasonable benefit/risk ratio applicable to any medical treatment. It will be understood that the total daily usage of the compositions of the present disclosure will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 30 mg/kg of body weight per day, for example about 1-30 mg/kg, or about 5-10 mg/kg.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (e.g., RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

It will be understood that each of the methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Other than in the operating examples, or where otherwise indicated, all numbers expressing quantities of ingredients or reaction conditions used herein should be understood as modified in all instances by the term “about.” The term “about” is used herein to mean approximately, roughly, around, or in the region of. When the term “about” is used in conjunction with a numerical range, it modifies that range by extending the boundaries above and below the numerical values set forth. The term “about” when used in connection with percentages will mean.+−0.1%.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example

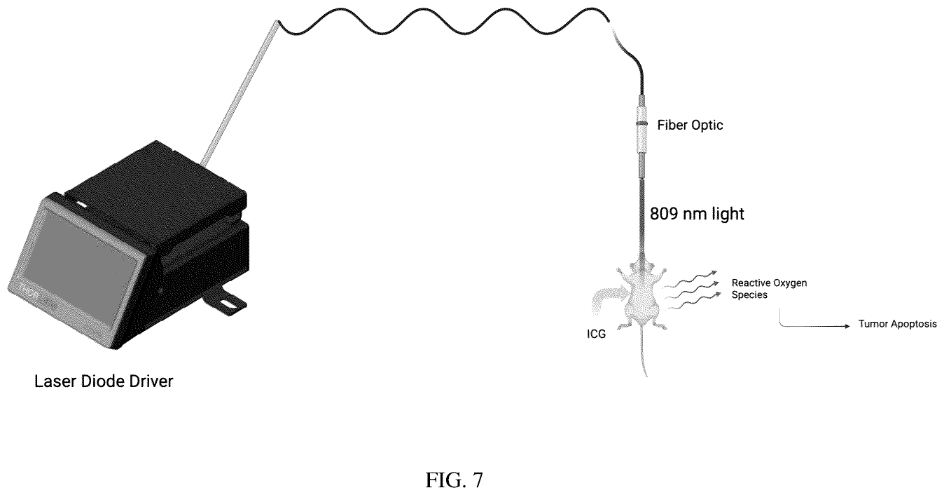

We set up a study of PDT with ICG using patient derived orthotopic xenograft (PDOX) mouse model (FIG. 7). In the PDOX model, the mouse liver has human liver cancer. After making this PDOX model, we administered ICG and found that ICG was retained in HCC only after 24 hours. At 0 hr, ICG was present in the whole body. At 6 hrs, ICG was present in the liver. At 24 hrs, ICG was confined in only HCC inside the liver. We then found the optimal laser wavelength that liver-ICG can absorb with maximum intensity which was 809 nm (FIG. 2). We set up the laser to treat HCC with PDT with the setting numbers in Table 1.

TABLE 1
Laser set-up for in vivo treatment (809 nm)

| | 30 J | 300 J |
|---|---|---|
| Laser machine | 0.1 W 300 sec → 30 J | 6 W 50 sec → 300 J |
| conditions | | |
| Actual reflected | ~25 mW 300 sec → ~7.5 J | ~2 W 50 sec → ~100 J |
| laser intensity | | |
| Distance between | 10 cm | |
| laser and subject | | |

| | |
|---|---|
| Laser diameter | 2.5 cm |
| Number of | Irradiate total of 3 times (once at |
| investigations | location of Supine, Right, Left) |

After applying the laser as above, we found breakdown of ICG at 24-48 hrs with IVIS imaging and found tumor necrosis on the MRI 2 weeks after treatment. On the pathologic evaluation, we found apoptosis of HCC and breakdown of ICG on the specimen.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for treating liver cancer in a patient in need thereof, comprising
administering at least one dose of a therapeutically effective amount of a composition comprising indocyanine green (ICG), wherein cells of the cancer that take up the ICG are sensitized to a photodynamic therapy (PDT) light source, and
applying a PDT light source to the liver of the patient, wherein the PDT light source comprises
a transarterial fiber optic coaxial catheter comprising a laser light,
a sheet having an array of light-emitting diode (LED) lights, or
a micro-injectable LED implant configured for delivery inside the liver,
wherein the PDT light source is applied for a time period sufficient to release reactive oxygen species from the photosensitizing agent and induce apoptosis of cancer cells.

2. The method of claim 1, wherein the ICG is bound to an additional photosensitizing agent.

3. The method of claim 1, wherein the pharmaceutical composition is administered intravenously or intraperitoneally.

4. The method of claim 1, wherein the PDT device is the transarterial fiber optic coaxial catheter.

5. The method of claim 1, wherein the laser light has a wavelength of 805-815 nm.

6. The method of claim 1, wherein the at least one dose of ICG is at least 5 mg/kg.

7. The method of claim 1, wherein the at least one dose of ICG is administered daily from one to five days.

8. The method of claim 1, wherein PDT is applied to the liver at least 24 hours after administration of a first dose of ICG.

9. The method of claim 1, wherein the liver cancer is hepatocellular carcinoma.

10. A photodynamic therapy (PDT) system, comprising
a composition comprising indocyanine green (ICG), and
a PDT device comprising at least one light source enabled for application of light to a target tumor, wherein the PDT device is
a transarterial fiber optic coaxial catheter comprising a laser light,
a sheet having an array of light-emitting diode (LED) lights, or
a micro-injectable LED implant configured for delivery inside the liver.

11. The PDT system of claim 10, wherein the at least one light source has a wavelength of 805-815 nm.
